(19) United States
(12) Patent Application Publication
(10) Pub. No.: US 2004/0133199 A1
(43) Pub. Date: Jul. 8, 2004

(54) External fixation device for reducing bone fractures

(76) Inventors: Michele Coati, San Pietro In Cariano (IT); Luigi Rossi, Peschiera Del Garda (IT)

Correspondence Address: AKERMAN SENTERFITT, P.O. BOX 3188, WEST PALM BEACH, FL, 33402-3188, US

(21) Appl. No.: 10/468,131

(22) PCT Filed: Mar. 4, 2002
(86) PCT No.: PCT/EP02/02311

(30) Foreign Application Priority Data
Mar. 5, 2001 (EP) 01830149.9

(51) Int. Cl. A61B 17/56

(52) U.S. Cl. 606/54

(57) ABSTRACT

The invention relates to a novel type of an external fixation device having a carrying structure (1; 40) of interconnected elements (2, 5, 6) comprising an extendible central body (2), and clamps (5, 6) for bone screws respectively articulated on opposed ends (3, 4) of the central body (2), said central body comprising two parts (8, 9) telescoping slidable one on the other, each of said parts (8, 9) including a first portion (8a; 9a) of elongate shape formed integrally with a second end portion (8b; 9b) of substantially cylindrical shape, said first portions (8a, 9a) of the parts (8, 9) being slidably coupled to each other by means of a driving groove (7) formed longitudinally in one the first portion (8a) and a corresponding slide (7a) formed longitudinally in the other first portion (9a) and means being provided in said parts (8, 9) for stopping their sliding movement characterized in that it further comprises at least one plate (27; 41; 42; 43; 60) having predetermined contour shape and thickness, at least one recess (28; 44; 47; 51) formed on the surface of at least one of the interconnected elements (2, 5, 6) and having its contour shape matching the contour shape of said at least one plate (27; 41; 42; 43; 60), and means (30, 31; 46, 46a; 49, 50 53, 54) for releasably securing said at least one plate (27; 41; 42; 43; 60) in said at least one recess (28; 44; 47; 51).

### TECHNICAL FIELD

This invention broadly relates to an external fixation device for reducing bone fractures in orthopedic surgery.

More particularly, the invention relates to an external fixation device having a carrying structure made up of interconnected elements, specifically a structure comprising an extendible central body and oppositely located clamps for bone screws, which clamps are articulated to respective ends of the central body by means of ball joints.

### BACKGROUND ART

In this specific technical field, external fixation devices have long been employed to foster recovery of bone fractures by holding the bone fragments firmly in place.

Such external fixation devices comprise a central body of substantially cylindrical shape, which is extendible axially and has bone screw clamps articulated to its opposed ends by means of ball joints. The clamps are connected to rod-like bone screws which have been implanted into the cortex of the broken bone, from either sides of the fracture. Usually, two or three screws are adequate to guarantee a broken bone.

A variety of fixation devices to cope with different types of fractures and traumatisms are normally available from their suppliers.

Thus, fixation devices for the tibia and the femur, fixation devices for the humerus, fixation devices for joints such as the ankle and the elbow, and fixation devices for the wrist are available.

A well-recognized demand in this technical field is that of providing the device with visually identifiable means which should assist the orthopedist in his/her work of placing the device to a fractured part of an individual's skeleton, and later, which should give a visual indication, even approximate, of the mutual positions of the working parts of the fixation device.

Another demand from this field is that of providing the fixation device with decorative elements effective to enhance the outward appearance of the fixation device, for the orthopedist's sake as well as the patient's sake.

Unfortunately, state-of-art fixation devices come short of filling the above demands in that they are made of a homogeneous structure, usually of a plastic material transparent to X-radiation to permit unobstructed radiographs of the fractured region. Thus, the outward appearance of such fixation devices is often so unsightly that they are hidden under some covering.

Furthermore, any improvements in the direction of filling the above demands are restrained by the relatively high manufacturing cost of fixation devices, especially those incorporating one or more elements made up of a material transparent to X-radiation, and by the need to maintain a desired transparency to X-radiation and biocompatibility of the used materials.

U.S. Pat. No. 5,662,648 discloses an auxiliary device for use in the external setting of fractures which is particularly adapted for use in association with an external fixator or splint having bolt-holding clamps with spherical joints for fixing bone bolts or screws which can be implanted in fractured pieces of bone.

The auxiliary device comprises an axially elongatable central body having coupling means at its ends for releasable attachment to corresponding bolt-holding clamps, the coupling means being connected to the longitudinal ends of the central body by means of a pair of hinges having axes which are parallel to each other and perpendicular to the longitudinal axis of the central body.

Particularly, each hinge is connected to an end portion by means of a spherical or ball joint formed in a ferrule which is provided with a reference mark applied on its outer surface so as to identify the position of the axis of hinge when the device is fully assembled.

However, a reference mark as above can be easily altered for instance by repeated sterilisation of the device in steam autoclaves or can chip away due to occasional impacts during use of the device.

U.S. Pat. No. 6,030,386 discloses an improved telescopically adjustable strut to be used in conjunction with an external orthopaedic fixator.

The strut includes a shaft having an annular recess for the attachment of an identification clip or band. The identification clip is resilient and encompasses more than 180 degrees of the recess such that it will snap onto the recessed portion of the shaft and may be removed and transferred to another similarly designed strut where necessary.

U.S. Pat. No. 4,671,916 discloses a method of identifying instruments belonging to a set, which comprises forming at least one bore in a location of each instrument and securing at least one coloured plug of a preferably autoclavable material in the bore.

The plug may have a distinctively-shaped head portion which protrudes outwardly of the instrument surface or may have multiple end locks at intermittent positions, the excess of which can be cut away.

A method of identifying instruments similar to that according to U.S. Pat. No. 4,671,916 is also disclosed in FR 2770127.

EP 0807419 discloses a compact external fixator for the treatment of fractures in small bones and in children which comprises a pair of clamps for bone bolts connected by means of corresponding ball joints to a central body comprising at least one male member and one femal member which are joined telescopically.

However, none of the above documents of the prior art from U.S. Pat. No. 6,030,386 to EP 0807419 disclose or suggest providing identifiable means on external fixation devices as defined above, which would assist the orthopaedist in his/her work connected to the use of those devices.

The underlying technical problem of this invention is to provide an external fixation device for stabilizing bone fractures, with structural and functional features appropriate to fill at least one of the aforesaid demands.

### DISCLOSURE OF INVENTION

The technical problem is solved according to the invention by an external fixation device as previously indicated and as defined in the characterizing portion of claim 1 and followings.

The features and advantages of the external fixation device according to the invention will be apparent from the following description of embodiments thereof, given by way of non-limitative examples with reference to the accompanying drawings.

### BRIEF DESCRIPTION OF DRAWINGS In the drawings:

FIG. 1 is a perspective view of a unilateral external fixation device according to this invention, shown in its closed setting;

FIG. 2 is a perspective view of the fixation device in FIG. 1, shown in its open setting;

FIG. 3 is an exploded perspective view of the fixation device shown in FIG. 1;

FIGS. 4 to 6 are side views, taken under different angles, of the fixation device shown in FIG. 2;

FIG. 7 is an unilateral axial type of external fixation device according to another embodiment of this invention;

FIGS. 7a and 7b are side views, taken under different angles, of the fixation device shown in FIG. 7;

FIG. 8 is a perspective view of a detail of the fixation device shown in FIG. 8;

FIG. 9 is an exploded perspective view of the detail shown in FIG. 8;

FIG. 10 is a perspective view of another detail of the fixation device shown in FIG. 7; and

FIG. 11 is an exploded perspective view of the detail shown in FIG. 10.

### MODES FOR CARRYING OUT THE INVENTION

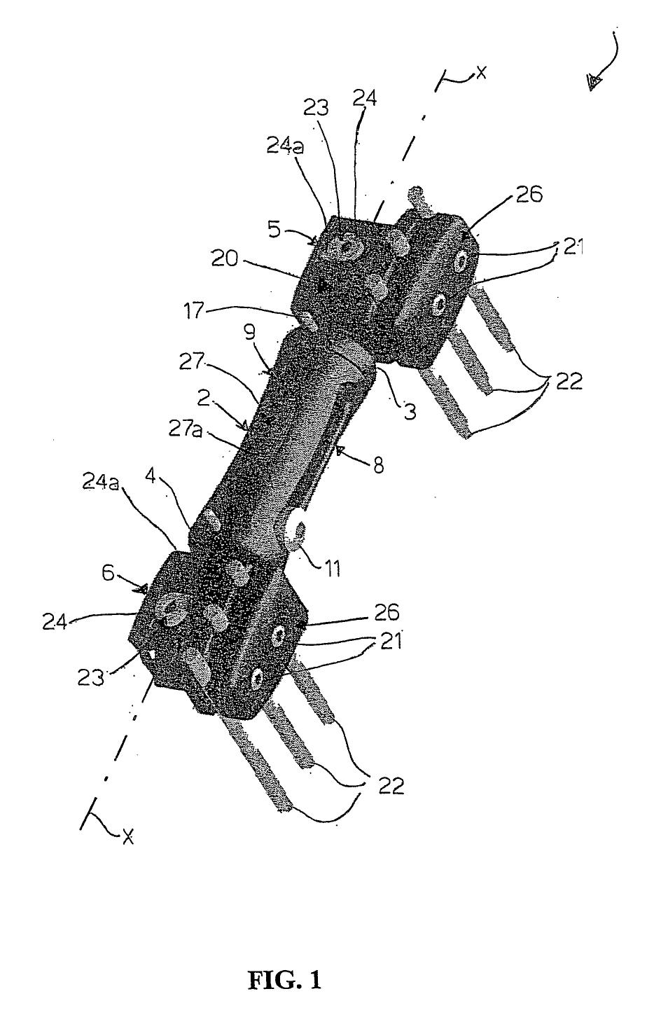

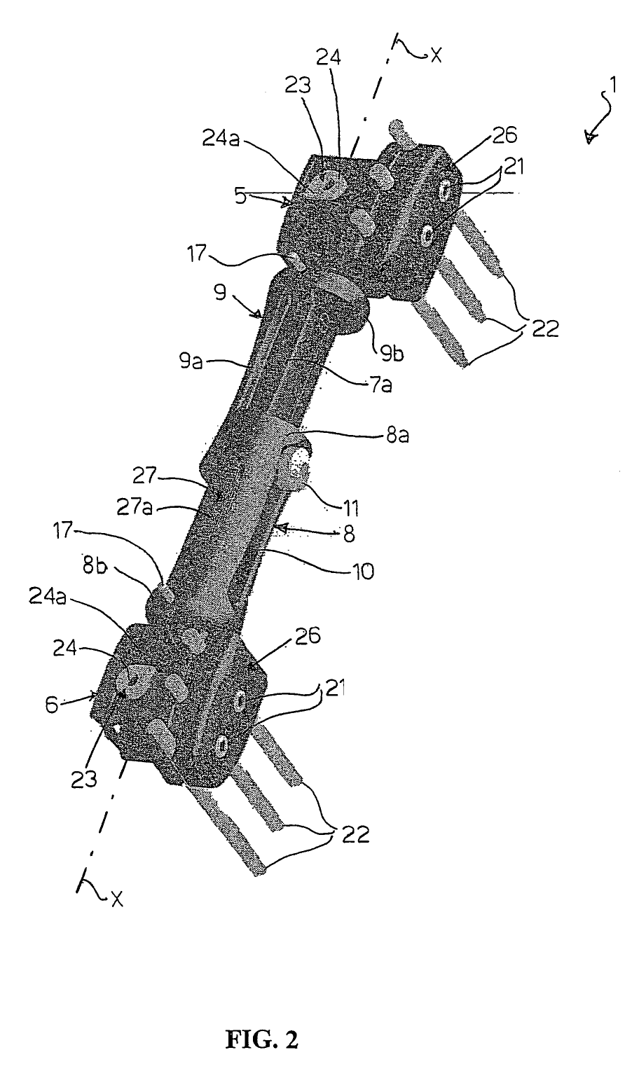

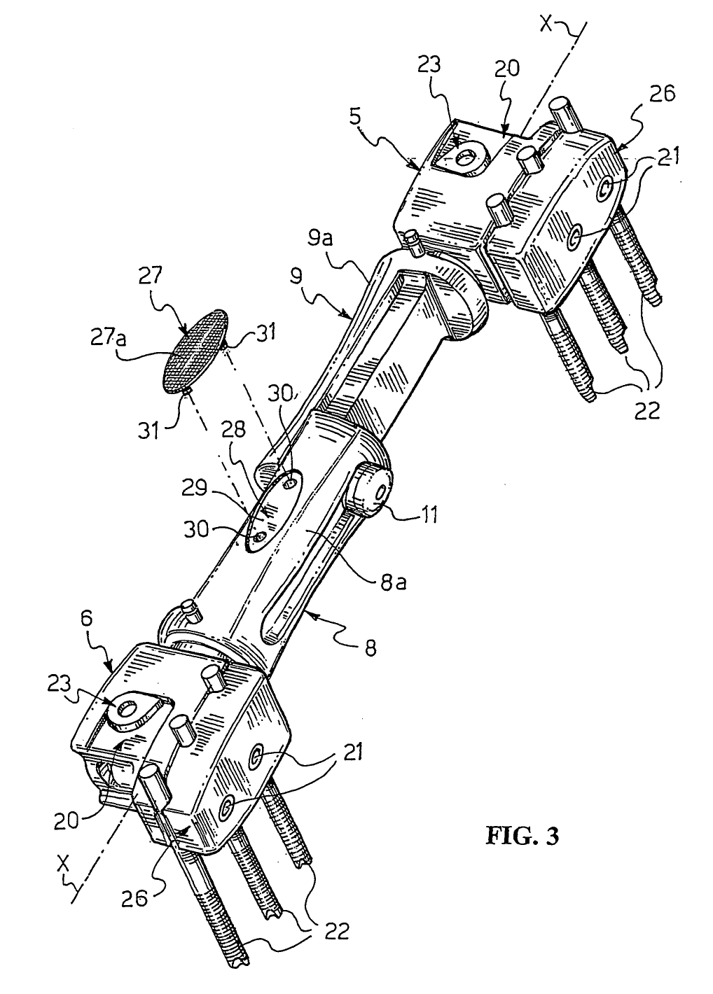

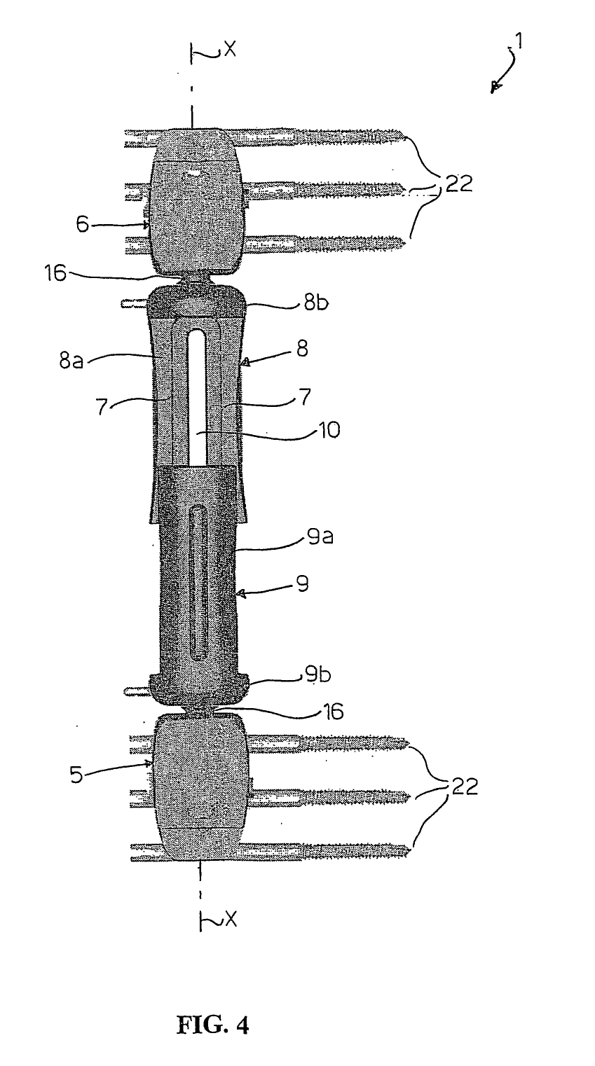

With reference to annexed FIGS. 1 to 6, a unilateral axial external fixation device for reducing bone fractures in orthopedic surgery, according to this invention, is shown generally at 1.

The fixation device 1 has a carrying structure made up of interconnected elements, which structure comprises a rod-like central body 2 having a longitudinal axis x-x, and having opposite ends 3, 4 which are articulated to respective clamps 5, 6 for bone screws.

The rod-like central body 2 and clamps 5, 6 are preferably made up of a material transparent to X-radiation, such as a polyetherketone plastics matrix, known as “Peek”, suitably reinforced with a predetermined amount of carbon fibers to confer a suitable rigidity.

The rod-like central body 2 is axially extendible and comprises a first part 8 and a second part 9 slidably coupled to each other together to allow the central body 2 to be extended telescopically.

Said parts 8, 9 comprise each a first portion 8a, 9a, of elongate shape, which is formed integrally with a second end portion 8b, 9b being substantially cylindrical in shape and shorter. Thus, each part 8, 9 is L-shaped in side view.

The elongate portions 8a, 9a are slidably coupled to each other by means of a driving groove 7, formed longitudinally in the portion 8a, and a corresponding slide 7a formed longitudinally in the other portion 9a.

The fixation device 1 further comprises conventional means for stopping said parts 8 and 9 in their sliding movement. This stopping means consists of a set screw 11 extending perpendicularly to the longitudinal axis x-x and having a threaded end portion screwed in a conventional manner in a threaded hole (not shown) provided in the slide 7a.

In addition, the portion 8a of the part 8 is formed with a clearance slot 10 which spans longitudinally most of said portion to let the screw 11 go through it preferably with a smooth section of its shank four easier sliding movement of the portions 8 and 9 one on the other.

The screw 11 is an Allen screw, for more convenient operation by a corresponding wrench.

The extension of the rod-like central body 2 of the fixation device 1 can be adjusted by loosening and tightening the screw 11, depending from the dimensions of the broken bone. No relative rotation is instead allowed of the parts 8 and 9 about the longitudinal axis x-x of the rod-like central body 2, due to the C- and T-shaped cross-sections of the driving groove and the slide.

In the setting of minimum bulk of the rod-like central body 2, the elongate portions 8a and 9a of the parts 8 and 9 result fully superposed, with one abutting the cylindrical end portion 9b, 8b of the other.

The opposed ends 3, 4 of the rod-like central body 2 are articulated to the respective clamps 5, 6 for bone screws by means of ball joints.

These ball joints are known per se, and are ball-and-socket joints 16 mounted to each clamp 5, 6.

In particular, each ball-and-socket joint 16 comprises a ball head with a shank, the ball head being fitted into a socket formed in each clamp 5, 6, and the shank being fitted, in a conventional manner, in a corresponding seat in each of the ends 3, 4 of the rod-like central body 2.

The shank is held in its seat by a conventional releasable retention means, e.g. a pin 17. This pin 17 also provides a reference base for a dynamization compressor, such as that disclosed in European Patent Application No. 0 734 233.

The clamps 5 and 6 comprise each a main body 20, in which the socket for receiving the ball head of the ball-and-socket joint 16 is formed.

A sideplate 26 is removably associated with the main body 20 for tightening the clamp onto bone screws 22, in the example three screws 22 lying in a plane parallel to the plane containing the axis x-x and being placed a predetermined distance away therefrom.

The fixation device 1 also comprises locking means 23 of the ball-and-socket joint 16 in a selected angular position. This locking means 23 comprises of a drive rotary shaft acting in the main body 20 and having a handling end 24 that protrudes from said main body 20.

The end 24 has a radial projection 24a for more convenient manipulation of the locking means 23.

The structural and operational characteristics of the clamps 5 and 6, of the locking means 23, of the sideplate 26, and of the releasable connection of the latter to the main body 20 are conventional, and will not be further discussed.

In accordance with the invention, the fixation device 1 has an oval plate 27 of a given thickness releasably attached to the rod-like central body 2.

In particular, the plate 27 is lodged into a corresponding seat consisting of a recess 28 formed on the surface of the elongate portion 8a of the rod-like central body 2. Preferably, the depth of the recess 28 is substantially equal to the thickness of the plate 27.

The plate 27 is made releasably fast with the elongate portion 8a, by fitting two opposed pins 31 integrally formed on the underside of the plate 27 into corresponding blind holes 30 formed in the bottom 29 of the recess 28.

Thus, the plate 27 is secured in the recess 28, but is easily removed for replacement.

Advantageously, the plate 27 is formed from a plastic material that is transparent to X-radiation and a contrasting colour with that of the rod-like body 2, so that the chromatic effect will distinguish the fixation device 1 of this invention.

However, this plate could be made of another plastics or aluminium alloy material producing low interference on X-radiation.

The plastic plate 27 is formed by conventional processes such as injection moulding, and can be either bulk-stained or post-moulding dyed by conventional methods.

Preferably, the plate 27 is made with a thin thickness, preferably comprised in the range of 0.5 to 1.5 mm, so that the plate will not appreciably interfere on a standard X-raying apparatus as a result of any opaque material to X-radiation entering its composition and/or because of the dyestuff.

Also, the plate 27 can bear, on its outer surface 27a exposed to view, such captions, insignia, logos and/or graphic compositions as may be the manufacturer's choice and/or demanded by the user, for example, in order to distinguish the fixation device 1 according to specific taste and personal preference.

In this embodiment, the visible outer surface 27a of the plate 27 is advantageously knurled to render more safe the grip and the manipulation of the fixation device 1.

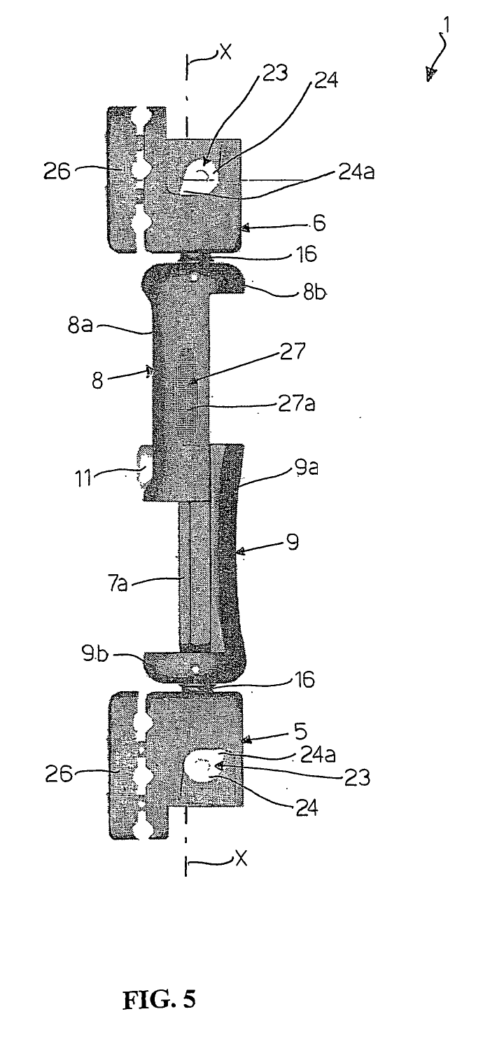

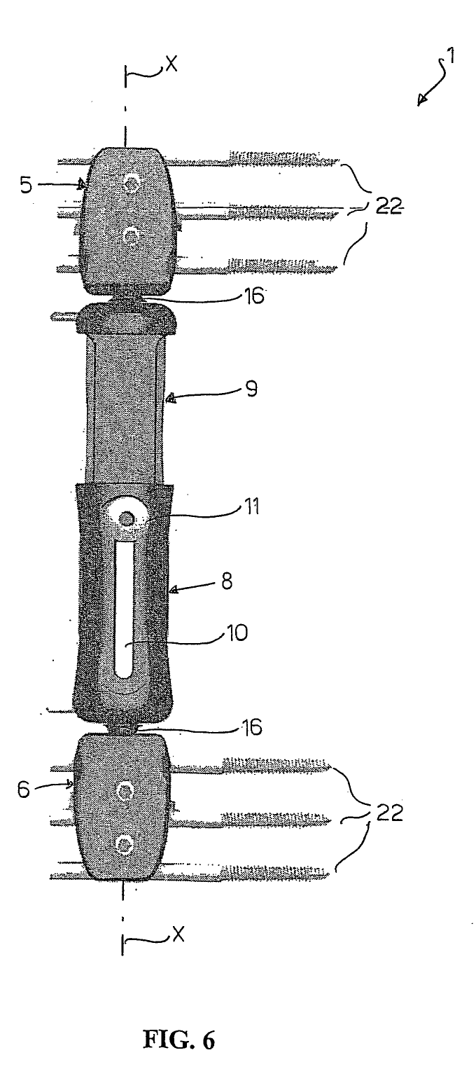

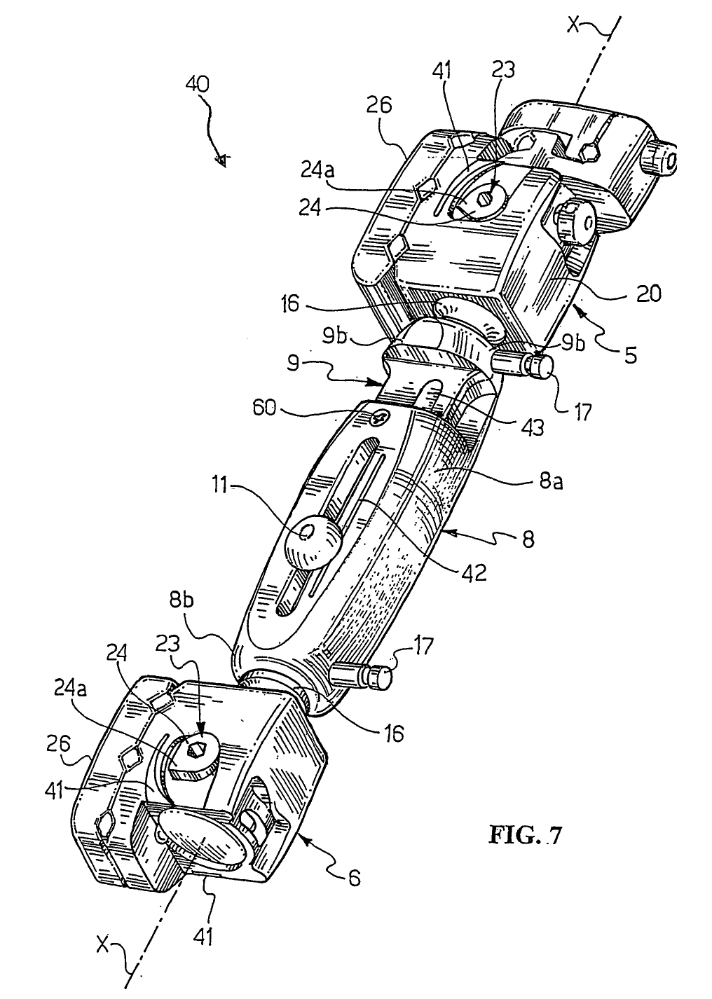

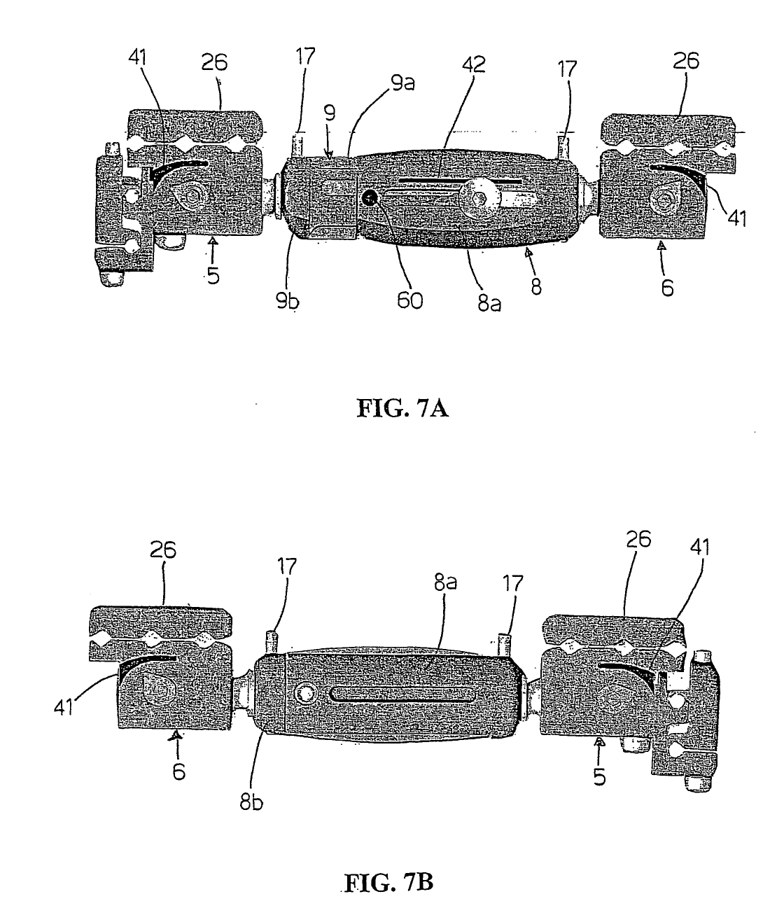

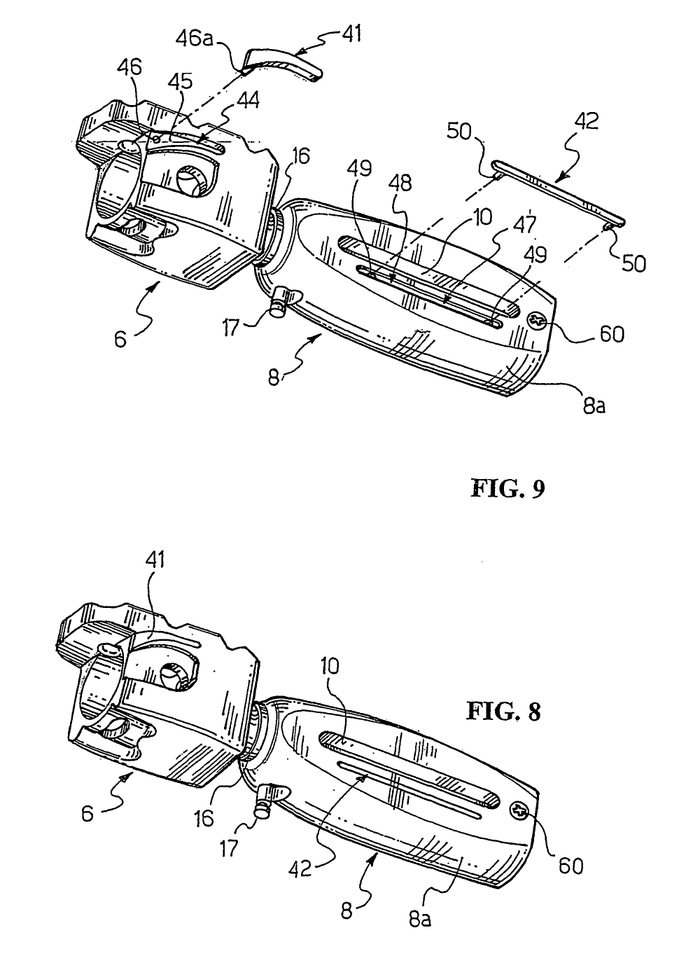

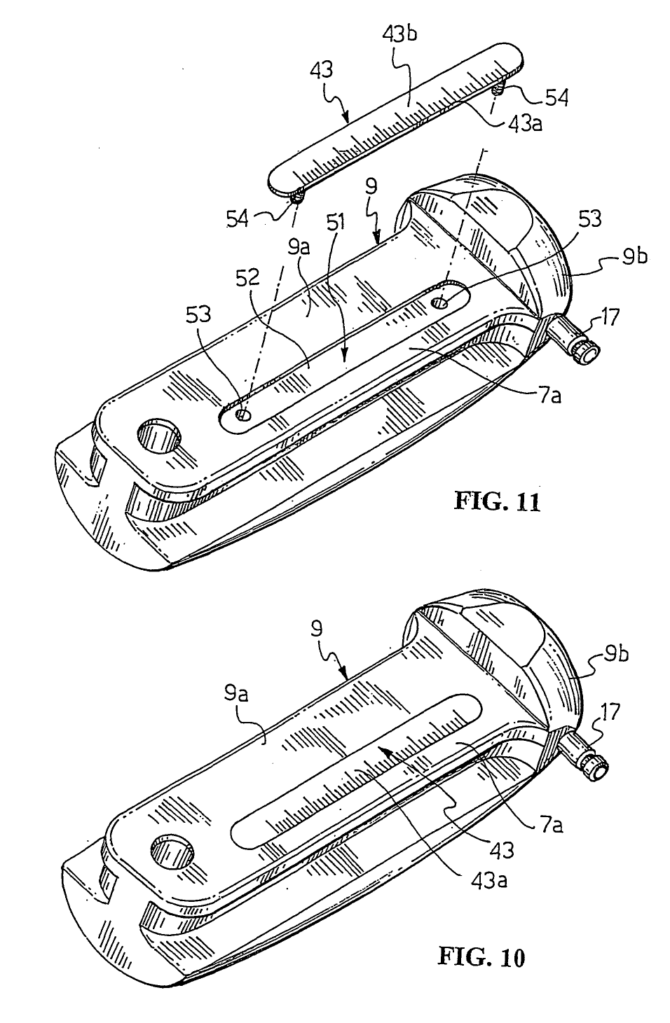

Making now reference to FIGS. 7 to 11, an external fixation device of the unilateral axial type, according to another embodiment of the invention, is shown generally at 40.

In all these Figures, elements having the same construction as or functionally equivalent to those of the fixation device 1 shown in FIGS. 1 to 6 are denoted by the same reference numerals.

The fixation device 40 has two plates 41 releasably attached to opposed sides of each clamp 5, 6, two plates 42 and 60 releasably attached to part 8 of the rod-like central body 2, and one plate 43 releasably attached to part 9 of the central body 2.

In particular, each plate 41 is placed close to the locking means 23 of the ball-and-socket joint 16 and extends between a substantially central location on one of the clamps 5, 6 and the free end thereof, so as to provide a visual indication of the movement of the radial projection 24a on the handling end 24 of said locking means 23.

In other words, each plate 41 situates in the background from the arcuate path travelled by the radial projection 24a as the end 24 of the locking means 23 is manipulated.

In practice, each plate 41 has a substantially comma-shaped contour with a cross-section that decreases gradually toward a central position of one of the clamps 5, 6.

Thus, each plate 41 advantageously provides the orthopedist with a visual indication of the rotation of the handling end 24, as well as a visual indication, albeit approximate, of the operational state of the locking means 23.

For example, a radial projection 24a aligned to regions of increasing cross-section of the plate 41 as a result of the handling end 24 having been manipulated, advantageously provides the orthopaedist with an indication of increased effectiveness of the ball-and-socket joint 16 locking.

Each plate 41 is lodged in a respective seat in one of the clamps 5, 6. This seat consists of a recess 44 having preferably a depth from the surface that is substantially equal to the thickness of a plate 41.

Each plate 41 is releasably fast to one of the clamps 5, 6 by fitting a pin 46a, integrally formed on the underside of the plate 41 into a corresponding blind hole 46 formed in the bottom 45 of a recess 44.

The plate 42 consists of an elongate bar extending lengthwise on the elongate portion 8a of the rod-like central body 2, laterally of the slot 10, to nearly span its full axial length.

In practice, the plate 42 is lodged in a corresponding seat provided in the elongate portion 8a of the rod-like central body 2. This seat consists of a recess 47 having preferably a depth from the surface that is substantially equal to the thickness of the plate 42.

The plate 42 is releasably fast to the elongate portion 8a by fitting two opposed pins 50, integrally formed on the underside of the plate 42 into respective blind holes 49 provided in the bottom 48 of the recess 47.

The plate 60 is circular in shape and locates centrally close to one end of the elongate portion 8a of the rod-like central body 2. The releasable attachment of the plate 60 to the elongate portion 8a is performed in a similar way as the plate 42, and therefore it will not be further described.

The plate 43 consists of an elongate bar extending lengthwise on the slide 7a formed in the portion 9a of the rod-like central body 2.

In practice, the plate 43 is lodged in a corresponding seat provided in said slide 7a. This seat consists of a recess 51 having preferably a depth from the surface that is substantially equal to the thickness of the plate 43.

The plate 43 is releasably fast to the slide 7a by fitting two opposed pins 54 integrally formed on the underside of the plate 43 in respective blind holes 53 provided in the bottom 52 of the recess 51.

In particular, the plate 43 has a visible outer surface 43b impressed with a calibrated scale 43a that advantageously provides the orthopedist with a measure of the extension of the rod-like central body 2 achieved by a sliding movement of the parts 8 and 9.

The plates 41, 42, 43 and 60 just described are advantageously made of a plastic material transparent to X-radiation and colored to create contrast with the color of the fixation device parts bearing them. Alternatively, these plates could be made of any other plastics or aluminum alloy material producing low interference on X-radiation.

Preferably, the plates 41, 42, 43 and 60 are made with a thin thickness preferably comprised in the range of 0.5 to 1.5 mm, so that they will not interfere appreciably on standard X-ray equipment.

The fixation device of this invention does solve the technical problem and offers a number of advantages, among which the foremost is its being highly versatile and quite simple to manufacture., In fact, according to the invention, any decorative element can be provided on the fixation device structure by exchanging plates of contrasting colors, rendering the plates interchangeable.

Another advantage of the fixation device according to the invention is that the plates can be made of the same material transparent to X-radiation as that of the other components of the device, so that the plates would not interfere on standard X-ray equipment, or would only interfere to a reduced extent by reason of the dyestuff they incorporate.

Furthermore, in the event of the plates being made of an plastic opaque to X-radiation or an aluminum alloy, their interference on the X-ray equipment can be suppressed or at least attenuated by making them very thin and/or placing them in unexposed areas of the device to X-radiation.

A further advantage of the fixation device according to the invention is that of its high level of bio-compatibility which is not altered by the presence of contrasting colour plates thereon.

Still another, no less important advantage of the fixation device according to the invention, is that the provision of decorating plates is of minor relevance to the manufacturing cost of the device. Consequently, the manufacturing cost of latter is fully comparable with that of similar fixation devices of the state of the art.

In order to satisfy contingent and specific requirements, a skilled person in the art could make, in a obvious way, several changes and modifications to the fixation device of this invention without departing from the scope of protection of the invention as defined by the following claims.

1. An external fixation device for reducing bone fractures having a carrying structure (1;40) of interconnected elements (2,5,6) comprising an extendible central body (2), and clamps (5,6) for bone screws respectively articulated on opposed ends (3,4) of the central body (2), said central body comprising two parts (8,9) telescoping slidable one on the other, each of said parts (8,9) including a first portion (8a;9a) of elongate shape formed integrally with a second end portion (8b;9b) of substantially cylindrical shape, said first portions (8a,9a) of the parts (8,9) being slidably coupled to each other by means of a driving groove (7) formed longitudinally in one the first portion (8a) and a corresponding slide (7a) formed longitudinally in the other first portion (9a) and means being provided in said parts (8,9) for stopping their sliding movement characterized in that it further comprises at least one plate (27;41;42;43;60) having predetermined contour shape and thickness, at least one recess (28;44;47;51) formed on the surface of at least one of the interconnected elements (2,5,6) and having its contour shape matching the contour shape of said at least one plate (27;41;42;43;60), and means (30,31;46,46a;49,50;53,54) for releasably securing said at least one plate (27;41;42;43;60) in said at least one recess (28;44;47;51).

2. A fixation device according to claim 1, in which the depth of said at least one recess (28;44;47;51) is substantially equal to the thickness of said at least one plate (27;41;42;43;60).

3. A fixation device according to either claim 1 or claim 2, in which said at least one plate (27;41;42;43;60) has a color contrasting with the color of said at least one of the interconnected elements (2,5,6) where said at least one recess (28;44;47;51) is formed.

4. A fixation device according to any of the preceding claims, in which said releasably securing means (30,31 ;46,46a;49,50;53,54) comprises at least one hole (30;46;49;53) formed in said at least one recess (28;44;47;51) and at least one pin (31;46a;50;54) formed integrally with said at least one plate (27;41;42;43;60) and fitted in said at least one hole (30;46;49;53).

5. A fixation device according to any of the preceding claims, in which at least one of the interconnected elements (2,5,6) is made of a material transparent to X-radiation.

6. A fixation device according to any of the preceding claims 1 to 4, in which said clamps (5,6) and said extendible central body (2) are made of a material transparent to X-radiation.

7. A fixation device according to claim 6, comprising at least one plate (27;42;60) releasably secured in at least one recess (28;47) formed in the surface of at least one of said first portions (8a,9a) of said central body (2).

8. A fixation device according to claim 7, comprising a plate (43) releasably secured in a recess (51) of said slide (7a), said plate (43) consisting of an elongate bar bearing a calibrated scale (43a) impressed in a visible outward surface (43b) thereof to provide a measure of the extension of the central body (2) produced by a sliding movement of the slidably assembled parts (8,9).

9. A fixation device according to claim 6, comprising at least one plate (41) releasably secured in at least one recess (44) formed in the surface of at least one of the clamps (5,6).

10. A fixation device according to any of the preceding claims, in which said at least one plate (27;41;42;43;60) has a multilineal contour shape.

11. A fixation device according to claim 10, in which said multilineal contour is oval, circular, or comma-shaped.

12. A fixation device according to any of the preceding claims, wherein said at least one plate (27;41;42;43;60) is made of a plastic transparent to X-radiation.

13. A fixation device according to any of claims 1 to 11, in which said at least one plate (27;41;42;43;60) is made of a material having a low interference with X-ray, preferably a plastic opaque to X-radiation or an aluminium alloy.

14. A fixation device according to any of the preceding claims, in which the thickness of said plate (27;41;42;43;60) is comprised in the range of 0.5 to 1.5 millimeters.
